(19) United States
(12) Patent Application Publication
(10) Pub. No.: US 2009/0253904 A1
(43) Pub. Date: Oct. 8, 2009

(54) Process for the Preparation of a Tetrahydro-1H-Azepines

(75) Inventor: Peter John Rosyk, Weinheim (DE)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: BOEHRINGER INGELHEIM PHARMA GMBH & CO. KG, Ingelheim (DE)

(21) Appl. No.: 12/373,936

(22) PCT Filed: Jul. 7, 2007
(86) PCT No.: PCT/EP07/57360

§ 371 Date: Apr. 9, 2009

(30) Foreign Application Priority Data
Jul. 18, 2006 (EP) 06117401.7

(51) Int. Cl. C07D 223/12

(52) U.S. Cl. 540/606

(57) ABSTRACT

The invention relates to an improved process for the preparation of a tetrahydro-1H-azepine of formula I

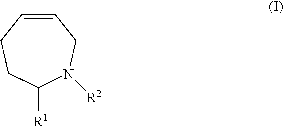

wherein R¹ and R² have the meaning given in the claims; by a ring closure metathesis of the corresponding diene of formula II

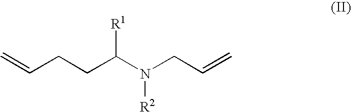

in the presence of a benzylidene ruthenium catalyst, wherein the phenyl group is substituted by a nitro group.

The invention relates to an improved process for the preparation of a tetrahydro-1H-azepine of formula I

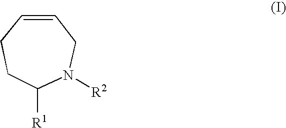

wherein R¹ and R² have the meaning given in the claims; by a ring closure metathesis of the corresponding diene of formula II

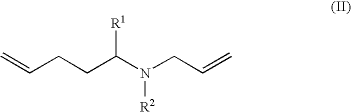

in the presence of a benzylidene ruthenium catalyst, wherein the phenyl group is substituted by a nitro group.

### BACKGROUND OF THE INVENTION

The tetrahydro-1H-azepines of formula I are valuable intermediates for the manufacture of relacatib (SB-462795, benzofuran-2-carboxylic Acid {(S)-3-methyl-1-[7-methyl-3-oxo-1-(pyridine-2-sulfonyl)azepan-4-ylcarbamoyl]butyl}amide) a Cathepsin-K (Cat-K) inhibitor, which can be orally administered in order to prevent bone metastases, cp. D. S. Yamashita et al., J. Med. Chem. 2006, 49, 1597-1612.

D. S. Yamashita et al. suggest to synthesize the tetrahydro-1H-azepines of formula I by a metathesis of the corresponding diene of formula II in the presence of the Hoyveda's catalyst, bis(tricyclohexylphosphine)benzylidene-ruthenium (IV) dichloride followed by column chromatography.

However, this process is not suitable for technical scale since the catalyst has to be used in comparably high amounts (0.01 to 0.1 equivalents of catalyst), which causes problems to isolate the desired product without ruthenium contamination. Furthermore, column chromatography is not applicable in a technical scale.

Surprisingly, it has been found that these problems can be avoided, if the tetrahydro-1H-azepines of formula I are prepared by a metathesis of the corresponding diene of formula II in the presence of a benzylidene ruthenium catalyst, wherein the phenyl group is substituted by a nitro group.

### SUMMARY OF THE INVENTION

Accordingly, the invention relates to a process for the preparation of a tetrahydro-1H-azepine of formula I

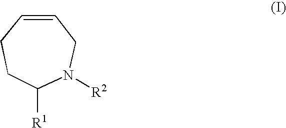

wherein
R¹ is a hydrogen atom or a C₁₋₆ alkyl group;
R² is a hydrogen atom, a pyridinesulfonyl or a protecting group,
which process comprises subjecting a diene compound of formula II

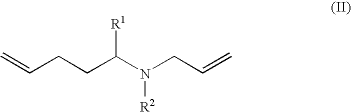

wherein R¹ and R² are as defined hereinbefore;
to a metathesis cyclisation reaction in the presence of a ruthenium catalyst of formula III:

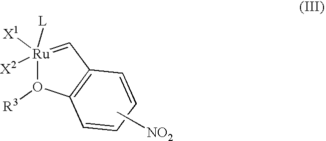

wherein
X¹ and X² each independently represent an anionic ligand;
L represents a neutral electron donor ligand; and
R³ represents a C₁₋₆ alkyl, C₂₋₆ alkenyl or C₆₋₁₂ aryl-C₁₋₁₆ alkyl group.

Another aspect of the invention is the use of a tetrahydro-1H-azepine of formula I prepared according to the process of this invention for the manufacture of a compound of formula IV

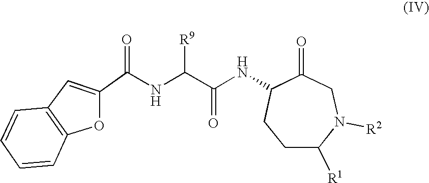

wherein R¹ and R² have the meaning given for formula I and
R⁹ represents a hydrogen atom or a C₁₋₆ alkyl group;
or a pharmaceutically acceptable salt thereof, in a manner known per se.

### DETAILED DESCRIPTION OF THE INVENTION

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆ alkyl means an alkyl group or radical having 1 to 6 carbon atoms. Unless otherwise specified below, conventional definitions of terms control and conventional stable atom valences are presumed and achieved in all formulas and groups.

By the term “C₁₋₆-alkyl” (including those which are part of other groups) are meant branched and unbranched alkyl groups with 1 to 6 carbon atoms, Examples of these include: methyl, ethyl, n-propyl, iso-propyl, n-butyl, iso-butyl, sec-butyl, tert-butyl, n-pentyl, iso-pentyl, neo-pentyl or hexyl. The abbreviations Me, Et, n-Pr, i-Pr, n-Bu, i-Bu, t-Bu, etc. may optionally also be used for the above-mentioned groups. Unless stated otherwise, the definitions propyl, butyl, pentyl and hexyl include all the possible isomeric forms of the groups in question. Thus, for example, propyl includes n-propyl and iso-propyl, butyl includes iso-butyl, sec-butyl and tert-butyl etc.

The term “C₂₋₆-alkenyl” (including those which are part of other groups) denotes branched and unbranched alkenyl groups with 2 to 6 carbon atoms. Examples include: ethenyl or vinyl, propenyl, butenyl, pentenyl, or hexenyl. Unless otherwise stated, the definitions propenyl, butenyl, pentenyl and hexenyl include all possible isomeric forms of the groups in question. Thus, for example, propenyl includes 1-propenyl and 2-propenyl, butenyl includes 1-, 2- and 3-butenyl, 1-methyl-1-propenyl, 1-methyl-2-propenyl etc.

The term “C₆₋₁₂ aryl” as used herein, either alone or in combination with another substituent, means either an aromatic monocyclic system containing 6 carbon atoms, being optionally substituted by 1 to 5 alkyl groups, which together have up to 6 carbon atoms, or an aromatic bicyclic system containing up to 12 carbon atoms. For example, aryl includes a phenyl, trimethylphenyl or a naphthyl-ring system.

The term “C₆₋₁₂ aryl-C₁₋₆ alkyl” as used herein, means either an aromatic monocyclic system containing 6 carbon atoms or an aromatic bicyclic system containing up to 12 carbon atoms, which is linked via an alkylene group having 1 to 6 carbon atoms. For example, C₆₋₁₂ aryl-C₁₋₆ alkyl includes a benzyl or a phenylethyl group.

In general, all tautomeric forms and isomeric forms and mixtures, whether individual geometric isomers or optical isomers or racemic or non-racemic mixtures of isomers, of a chemical structure or compound is intended, unless the specific stereochemistry or isomeric form is specifically indicated in the compound name or structure.

The term “pharmaceutically acceptable salt” as used herein includes those derived from pharmaceutically acceptable bases. Examples of suitable bases include choline, ethanolamine and ethylenediamine. Na⁺, K⁺, and Ca⁺⁺ salts are also contemplated to be within the scope of the invention (also see Pharmaceutical salts, Birge, S. M. et al., J. Pharm. Sci., (1977), 66, 1-19, incorporated herein by reference).

Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Synthetic Examples section.

Preferred is a process for the preparation of the compound of formula I from a diene of formula II, wherein a catalyst of formula III is employed, in which
L is a trihydrocarbylphosphine group, preferably a tri-(C₁₋₆ alkyl)-phosphine or a tri-(C₃₋₈ cycloalkyl)-phospine group, in particular a tricyclohexylphosphine group; or a group of formula

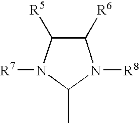

wherein
R⁵ and R⁶ each independently represent a hydrogen atom or a C₁₋₆ alkyl, C₂₋₆ alkenyl, C₆₋₁₂ aryl or C₆₋₁₂ aryl-C₁₋₆ alkyl group, preferably a hydrogen atom; or
R⁵ and R⁶ together form a double bond; and
R⁷ and R⁸ each independently represent a hydrogen atom or a C₁₋₆ alkyl, C₂₋₆ alkenyl, C₆₋₁₂ aryl or C₆₋₁₂ aryl-C₁₋₆ alkyl group, preferably a phenyl group which may be substituted by one, two or three groups selected from halogen atom, C₁₋₆alkyl and C₁₋₆alkoxy groups;
X¹ and X² each independently represent a halogen atom, preferably a chlorine atom; and
R⁴ represents a C₁₋₆ alkyl group, preferably a branched C₃₋₆ alkyl group.

More preferred are ruthenium catalysts of formula II, wherein the nitro group is attached in the para-position with respect to the point of attachment of the alkoxy group R³—O—.

Particularly preferred is a process for the preparation of a compound of formula I, wherein the ruthenium catalyst is a compound of formula IIIA

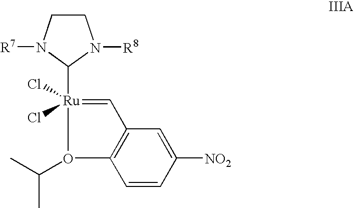

wherein R⁷ and R³ represent a trimethylphenyl group, in particular mesityl group.

Furthermore preferred is a process for the preparation of a compound of formula I according to the present invention, wherein the metathesis reaction is carried out in the presence of a diluent in a temperature range from 40 to 120° C., preferably from 60 to 100° C., in particular at about 80° C.

In another preferred embodiment of the present invention the methathesis reaction is carried out in the presence of a diluent selected from the group consisting of alkanes, such as n-pentane, n-hexane or n-heptane, aromatic hydrocarbons, such as benzene, toluene or xylene, and chlorinated hydrocarbons such as dichloromethane, trichloromethane, tetrachloromethane or dichloroethane.

Furthermore preferred is a process for the preparation of a compound of formula I, wherein the molar ratio of the diene compound of formula II to the catalyst of formula III ranges from 1000:1 to 150:1, preferably from 750:1 to 200:1, in particular from 600:1 to 300:1.

As a rule the process for the preparation of a compound of formula I is carried out at a ratio of the diene compound of formula II to diluent in the range from 1:400 by weight to 1:25 by weight, preferably from 1:200 by weight to 1:50 by weight, in particular from 1:150 by weight to 1:75 by weight.

The product obtained may be converted into the corresponding acid addition salt by known methods, by treating with an inorganic or organic acid. Preferably for this purpose the free base is taken up in a polar solvent, preferably an alcohol such as for example methanol, ethanol or isopropanol, water or a mixture thereof, particularly a mixture of ethanol and water and adjusted to a slightly basic pH with the corresponding acid, preferably an inorganic acid such as hydrochloric acid or sulphuric acid.

The aqueous phase is separated off, and made strongly alkaline, preferably to a pH of 10.0 to 14.0, particularly about 12.7, with a suitable base, preferably an alkali metal hydroxide, particularly sodium hydroxide solution. The aqueous phase thus obtained is extracted with a water-immiscible solvent, preferably an optionally halogenated, aliphatic or aromatic hydrocarbon, particularly toluene or dichloromethane. The combined organic phases thus obtained are concentrated. In this way the compound of formula I is obtained in the form of the free base.

The tetrahydro-1H-azepines of formula I, which have been prepared as described hereinabove can be used for the manufacture of a compound of formula IV, preferably a compound of formula IVA

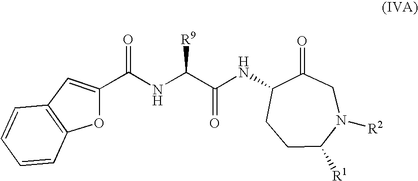

wherein R¹ and R² have the meaning given for formula I and R⁹ represents a hydrogen atom or a C₁₋₆ alkyl group;
in particular wherein
R¹ is a methyl group,
R² is a pyridine-2-sulfonyl group, and
R⁹ represents a 2-methylpropyl group;
most preferably for the manufacture relacatib.

### Example

Step a Preparation of the Catalyst 3a—The ruthenium catalyst is prepared in accordance with the method disclosed by K. Grela et al., Angew. Chem. Int. Ed. 2002, 41, No. 21 pp. 4038-4040 or EP 1554294, the disclosures of which is incorporated by reference in its entirety, in particular Scheme 2 on page 4038, and the experimental section describing the synthesis of compound no. 9 on page 4040.

THP Solution—23.5 g Tetrakishydroxymethylphosphoniumchlorid (80%, 98.7 mmol) is dissolved in isopropanol (35 ml) under a nitrogen atmosphere. Then 12.1 g (98.7 mmol) of a 45% KOH solution is added within 5 min while the solution is cooled (temperature 20-25° C.). After stirring the suspension for another 30 min under nitrogen, the mixture is filtered and the inorganic residue is washed with 20 ml of degassed isopropanol. The combined isopropanol solution is stored under a nitrogen atmosphere until use.

2-Methyl-2,3,4,7-tetrahydro-1Hazepine-1-carboxylic acid Benzyl Ester—750 ml of toluene are degassed by bubbling through nitrogen. 6.8 g (25 mmol) of N-allyl-N-(1-methylpent-4-enyl)carbamic acid benzyl ester are dissolved in 25 ml of degassed toluene and added into the reaction flask. The solution is heated up to 80° C. and 0.032 g (0.048 mmol) of the freshly prepared catalyst 3a is added under nitrogen in four portions over a period of 3 hours. After cooling to 60° C. 5.7 g (7 mmol) of the THP Solution is added to the reaction mixture. After stirring for 5 h at 60° C. the mixture is cooled to room temperature and extracted twice with 50 ml of degassed water, 50 ml of 0.5 M HCl, 50 ml of 0.5 M NaHCO3 solution, and 50 ml of water. Approx. 695 ml of toluene are distilled of at 50° C. in vacuo (150 mbar) and the residue is treated at 50° C. with 1.4 g of charcoal (Acticarbon L2S). After filtering off the charcoal the remaining liquid is slowly stirred into a mixture of 100 ml of water and 5 ml concentrated sulphuric acid. After 45 minutes' stirring at ambient temperature the phases are separated, and the aqueous phase is combined with 150 ml dichloromethane and adjusted to pH 12.9 with sodium hydroxide solution, the phases are separated and the aqueous phase is extracted three times with 150 ml dichloromethane. The combined organic phases are concentrated in vacuo to give the title compound (5.6 g, 91% yield):

¹H NMR (CDCl₃) δ 7.35-7.20 (m, 5H), 5.65-5.60 (1H, m), 5.15-5.12 (m, 2H), 4.45-4.05 (m, 2H), 3.63-5.57 (m, 1H), 2.25-2.10 (m, 2H), 1.90-1.60 (m, 2H), 1.15-1.12 (m, 3H); MS (ESI) 246.2 (M+H)⁺.

The title compound can be used to prepare relacatib as described by D. S. Yamashita et al., J. Med. Chem. 2006, 49, 1597-1612, the disclosures of which is incorporated by reference in its entirety, in particular Scheme 3 on page 1600, and the experimental section describing the synthesis of compounds nos. 10 to 13 and 44 to 58 on pages 1608 to 1610.

1. A process for the preparation of a tetrahydro-1H-azepine of formula I 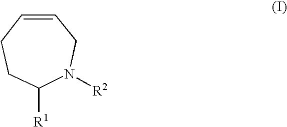
wherein
R¹ is a hydrogen atom or a C₁₋₆ alkyl group;
R² is a hydrogen atom, a pyridinesulfonyl or a protecting group,
which process comprises subjecting a diene compound of formula II 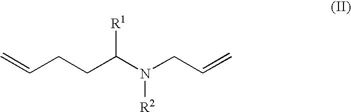
wherein R¹ and R² are as defined for formula I;
to a metathesis cyclisation reaction in the presence of a ruthenium catalyst of formula III: 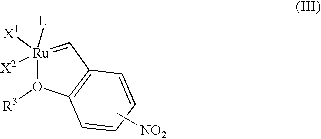
wherein
X¹ and X² each independently represent an anionic ligand;
L represents a neutral electron donor ligand; and
R³ represents a C₁₋₆ alkyl, C₂₋₆ alkenyl or C₆₋₁₂ aryl-C₁₋₆ alkyl group.

2. A process according to claim 1 for the preparation of a tetrahydro-1H-azepine of formula I, wherein L of formula III is a trihydrocarbylphosphine group or a group of formula 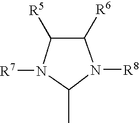
wherein
R⁵ and R⁶ each independently represent a hydrogen atom or a C₁₋₆ alkyl, C₂₋₆ alkenyl, C₆₋₁₂ aryl or C₆₋₁₂ aryl-C₁₋₆ alkyl group; or
R⁵ and R⁶ together form a double bond; and
R⁷ and R⁸ each independently represent a hydrogen atom or a C₁₋₆ alkyl, C₂₋₆ alkenyl, C₆₋₁₂ aryl or C₆₋₁₂ aryl-C₁₋₆ alkyl group;
X¹ and X² each independently represent a halogen atom; and
R³ represents a C₁₋₆ alkyl group.

3. A process according to claim 1 for the preparation of a tetrahydro-1H-azepine of formula I, wherein the ruthenium catalyst is a compound of formula IIIA 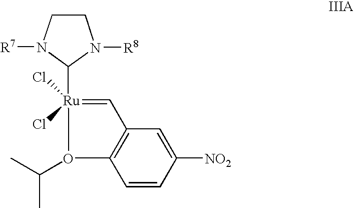
wherein both R⁷ and R⁸ represent a mesityl group.

4. A process according to claim 1 for the preparation of a tetrahydro-1H-azepine of formula I, wherein R¹ is a methyl group.

5. A process according to claim 1 for the preparation of a tetrahydro-1H-azepine of formula I, wherein metathesis reaction is carried out in the presence of a diluent in a temperature range from 40 to 120° C.

6. A process according to claim 1 for the preparation of a tetrahydro-1H-azepine of formula I, wherein metathesis reaction is carried out in the presence of a diluent selected from the group consisting of alkanes, aromatic hydrocarbons, chlorinated hydrocarbons.

7. A process according to claim 1 for the preparation of a tetrahydro-1H-azepine of formula I, wherein the molar ratio of the diene compound of formula II to catalyst of formula III ranges from 1000:1 to 150:1.

8. A process according to claim 1 for the preparation of a tetrahydro-1H-azepine of formula I, wherein the ratio of the diene compound of formula II to diluent ranges from 1:400 by weight to 1:25 by weight.

9. (canceled)

10. (canceled)

11. (canceled)
